(19) United States
(12) Patent Application Publication
(10) Pub. No.: US 2009/0088606 A1
(43) Pub. Date: Apr. 2, 2009

(54) SYSTEMS AND METHODS FOR PATIENT SPECIFIC ADAPTABLE TELEMONITORING ALERTS

(76) Inventors: Paul E. Cuddihy, Ballston Lake, NY (US); Mark D. Osborn, Castleton, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/863,529

(22) Filed: Sep. 28, 2007

(51) Int. Cl. A61B 5/00

(52) U.S. Cl. 600/300

(57) ABSTRACT

A system and method for determining a reference baseline of a patient and measuring trend shifts in physiological data to generate alerts acquires and receives physiological data from a patient under observation. Initial physiological data is received during a lock-in period and monitored physiological data is received during a diagnosis period. Shifts in the physiological data are measured by comparing the monitored physiological data to the initial physiological data. An alert is generated for shifts that exceed at least one of a pre-determined size and rate (i.e., a “threshold value”).

### BACKGROUND OF THE INVENTION

The present invention relates in general to automated collection and analysis of physiological data via telemonitoring, and, in particular, to a system and method for determining a reference baseline of a patient and measuring trend shifts in the physiological data to generate alerts.

Remote patient health monitoring (i.e., telemonitoring) refers to the use of telecommunications and information technology for purposes of monitoring patient health care. Telemonitoring is achieving a large rate of growth in many countries, due to several factors: the preoccupation in driving down the costs of health care, an increase in the number of aging and chronically ill population, and the increase in coverage of health care to distant, rural, small or sparsely populated regions. Among its many benefits, telemonitoring can help to solve increasing shortages of qualified health care providers, reduce distances and save travel time, and keep patients out of the hospital. Additionally, telemonitoring allows for patients who who have chronic ailments, such as chronic obstructive pulmonary disease, diabetes, congestive heart disease, or other disabilitating diseases, to stay at home and be monitored regularly by a remotely located healthcare professional.

Assessments on the ongoing condition of a patient can be made by way of physiological data gathered on-site with the patient. This data, which typically is comprised of physiological parameters such as heart rate, blood pressure, weight, and blood oxygen levels, is acquired and transmitted to a remote healthcare facility for subsequent analysis. The data acquired is typically analyzed in an automated fashion and feedback is provided to a healthcare provider.

For particular ailments such as diabetes, hypertension, and congestive heart failure, the condition of a patient can change rapidly. Thus, it is important for telemonitoring systems to provide an alert mechanism that notifies a healthcare provider of any potential problems that may be likely to develop in a patient before such a potential problem actually develops to a serious state. Typically, such alerts are generated when the telemonitoring system measures a physiological parameter that crosses some pre-defined, fixed threshold that has been set by a healthcare provider. For example, if a patient's blood pressure drops below or rises above a specified value (e.g., diastolic BP>90), the healthcare provider is notified of such an occurrence by an alert that is generated by the telemonitoring system.

Existing alert mechanisms, however, can create too many false alarms, causing a clinical staff to have to check-in on the patient when in fact nothing is wrong. These false alarms are typically the result of the type of alert used in the telemonitoring system. One such alert is a population alert, in which a single fixed threshold value for a specified physiological parameter is commonly used in monitoring all patients of a given population. A second type of alert commonly implemented is a patient specific alert, in which various patient specific parameters are considered when generating the fixed alert thresholds. In both cases, however, a large number of false alarms can still be generated due to a single acquired physiological measurement from a patient falling outside the pre-defined fixed thresholds. While damping techniques requiring two or three consecutive threshold exceedances have been implemented into telemonitoring systems employing population and patient specific fixed thresholds to reduce the number of false alarms, such damping techniques still do not adequately solve the problem.

In addition to the problem of false alarms generated by existing telemonitoring systems, such systems also include other limitations. For example, a healthcare provider may wish to be made aware of (e.g., via an alert) whether a patient may be trending into an area of potential medical concern, without having actually crossed a pre-defined fixed threshold. With existing telemonitoring systems, this is not possible.

It would therefore be desirable to have a system and method capable of reducing the number of false alarms generated by a telemonitoring system. It would also be desirable to have a system and method capable of measuring trends in acquired patient data and providing an alert if such trends are determined to cause potential medical concern, even if a fixed threshold has not yet been crossed.

### BRIEF DESCRIPTION OF THE INVENTION

The present invention provides systems and methods for telemonitoring physiological parameters of a patient that overcome the aforementioned drawbacks. Physiological data is acquired and used to determine a current patient status. Trend shifts and variations in further acquired physiological data are measured via the use of statistical algorithms and an alert is provided if such trends/variations cross a pre-determined threshold or are determined to cause potential medical concern.

In accordance with one aspect of the invention, a telemonitoring alert system for patient care includes a monitoring device configured to acquire physiological data from a patient under observation and a computer in communication with the monitoring device to receive physiological data therefrom. The computer is programmed to receive initial physiological data from the monitoring device during a lock-in period and receive monitored physiological data from the monitoring device during a diagnosis period. The computer is further programmed to measure shifts in the physiological data by comparing the monitored physiological data to the initial physiological data and generate an alert for shifts that exceed at least one of a pre-determined size and rate.

In accordance with another aspect of the invention, a method for telemonitoring a patient includes the steps of acquiring reference physiological data from a patient under observation and determining a reference baseline from the reference physiological data. The method also includes the steps of acquiring follow-up physiological data from the patient, comparing the follow-up physiological data to the reference baseline to identify a variation between the follow-up physiological data and the reference baseline, and generating an alert if the variation is outside a pre-determined threshold.

In accordance with yet another aspect of the invention, a computer readable storage medium includes a computer program thereon that provides alerts in a patient telemonitoring system. The computer program comprises a set of instructions that, when executed by a computer, causes the computer to receive physiological data on at least one physiological parameter, wherein the physiological data includes a first data set and a second data set. The set of instructions further causes the computer to determine a baseline patient status from at least a portion of the first data set, measure a shift in the second data set from the baseline patient status, and generate an alarm if the shift in the second data set exceeds a pre-determined threshold amount.

Various other features and advantages of the present invention will be made apparent from the following detailed description and the drawings.

### BRIEF DESCRIPTION OF THE DRAWINGS

The drawings illustrate one preferred embodiment presently contemplated for carrying out the invention.

In the drawings:

FIG. 1 is a schematic block diagram of a telemonitoring system incorporating the present invention.

FIG. 2 is a flow chart of a computer implemented process for analyzing physiological data acquired from a patient and for generating an alarm, useable with the system of FIG. 1.

FIG. 3 is a graphical representation of a weight telemonitoring simulation performed by telemonitoring system of FIG. 1.

### DETAILED DESCRIPTION OF THE PREFERRED EMBODIMENT

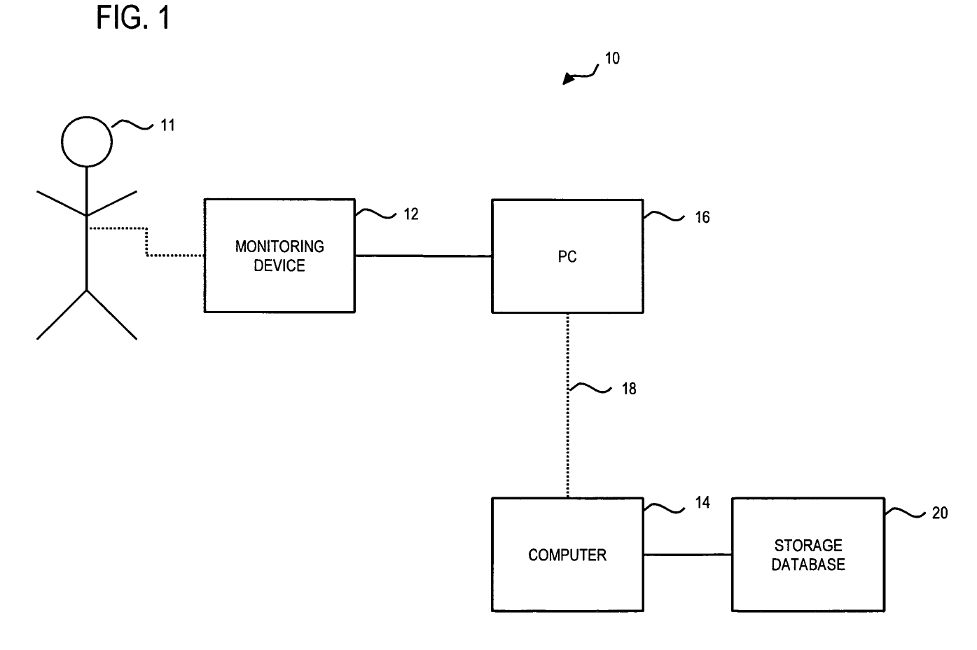

Referring to FIG. 1, a block diagram shows a telemonitoring system 10 for use with the present invention. Telemonitoring system 10 includes a monitoring device 12 that is located on-site with a patient 11 to be monitored. Monitoring device 12 can be configured to automatically measure physiological parameters of the patient 11, or alternatively, can be in the form of a device that allows for the patient 11 to perform manual self-check tests. While shown as a single device, monitoring device 12 can encompass a plurality of devices, each of which measures a specific physiological parameter. The physiological parameter(s) acquired by monitoring device(s) can include, but are not limited to, blood pressure, weight, pulse, and saturation of peripheral oxygen (Sp_{O2}).

The monitoring device 12, whether configured for automatic or manual measuring, acquires physiological data from the patient preferably at specified times that are separated by approximately regular intervals. Such regular intervals between data acquisition allows for the physiological data to be plotted and analyzed as a time series of data, as will be explained in greater detail below. The monitoring device 12 can also include circuitry for recording into a short-term, volatile memory the physiological data acquired thereby.

The physiological data stored in the monitoring device 12 is transmitted therefrom to a remotely located computer 14 or processing/server system located at a healthcare facility for further evaluation. Transmission of the physiological data can occur via any of a plurality of well-known methods. That is, data may be transferred from monitoring device 12 to a personal computer 16 located on-site with the patient and interfaced with the monitoring device. The PC 16 can then transfer the acquired physiological data to computer and/or server system 14 located at a designated healthcare facility via a communications medium 18 such as the Internet or telephone networks. Alternatively, the monitoring device 12 can be configured to send data directly therefrom, or a home hub may be utilized. Regardless of the exact manner of transfer of physiological data, the physiological data received by computer 14 at the healthcare facility is then stored in an electronic database 20 containing a patient profile. As will be described in greater detail below, computer 14 includes a computer readable storage medium having a computer program thereon that processes the incoming physiological data to provide data analysis and generate system alerts regarding a status of the patient.

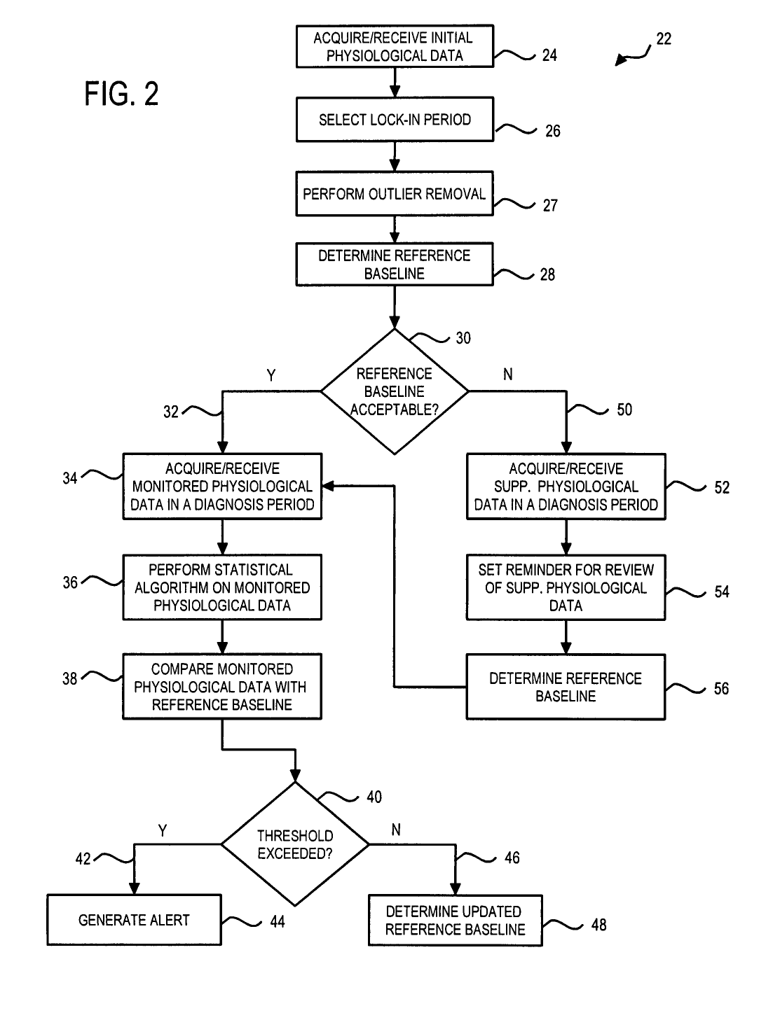

Referring now to FIG. 2, a computer implemented process 22 is shown that is performed by computer 14 (shown in FIG. 1) for analyzing the physiological data acquired from the patient and for generating an alarm. The process begins with receipt 24 of initial physiological data (i.e., reference physiological data, first data set) from a monitoring device representative of a measured physiological parameter of the patient under observation. The initial physiological data is gathered over a period of days or months, and at least a part of this data is used for determining a reference baseline (i.e., patient status baseline). That is, a floating “lock-in” period or long-term window is selected 26 covering at least a portion of the period of time during which initial physiological data was gathered (e.g., 90 days), and physiological data from this lock-in period is used in determining a reference baseline. The lock-in period chosen for establishing the reference baseline is determined by a healthcare provider and can, at least in part, be based on various factors such as the patient condition being monitored, the severity of this condition, and various additional factors that allow for optimized monitoring of the patient and accurate system alerts.

Prior to establishment of the reference baseline, the physiological data from the selected long-term window is examined and removal of outlier data 27 is performed. That is, in the determination of the reference baseline, certain initial physiological data may be excluded, such as minimum and maximum value readings present in the initial physiological data. The outlier removal process can be driven by statistics or by simple rules, and is performed in one of any well-known techniques. As an example, a fluctuation in weight of a patient of 15 lbs in one day and a return to the prior weight the next day would likely trigger removal of such data in the outlier removal process.

The determining 28 of the reference baseline is achieved through statistical algorithms that are performed by computer 14 to provide statistical analysis of the initial physiological data acquired during the lock-in period. The statistical analysis performed on the initial physiological data can vary, but can include calculation of a mean, a standard deviation, a variance, a range, an interquartile range, a moving average value, and an average absolute deviation. As will be explained in greater detail below, the reference baseline obtained via statistical analysis of the initial physiological data acquired in the lock-in period is compared with monitored physiological data acquired from the patient during a more recent short-term window diagnosis period.

Upon establishment of a reference baseline, an assessment 30 is made as to whether the reference baseline is acceptable. If the reference baseline is acceptable 32 and is indicative of a current state of a patient and of a “normal” reading for various physiological parameters, physiological data acquired thereafter (and referred to herein as monitored physiological data, follow-up physiological data, or a second data set) may be analyzed and monitored to predict and diagnose a change in the patient's condition. More specifically, monitored physiological data acquired and received during a recent diagnosis period or short-term window 34, the length of which is selected and defined by a healthcare professional (e.g., 3 days), is compared to the initial physiological data corresponding to the selected long-term window and contained in the reference baseline. As compared to telemonitoring systems where a singular reading of a physiological parameter may trigger a system alarm by being outside an acceptable threshold, telemonitoring system 10 and computer 14 (shown in FIG. 1) are configured to analyze the entirety of the physiological data acquired during a recent diagnosis period and perform statistical analysis of this monitored physiological data to determine overall trend shifts (i.e., variations) of the monitored physiological data as compared to the established reference baseline determined from the lock-in window.

Before comparison of the monitored physiological data to the reference baseline, an outlier removal process such as the one previously described above can be performed to remove erroneous data. Statistical algorithms are then performed 36 on the data (minus the removed outlier values) by the computer 14 to provide a statistical analysis of the monitored physiological data. Similar to the statistical analysis performed on the initial physiological data when establishing the reference baseline, the statistical algorithms performed on the monitored physiological data can provide calculation of a mean, a standard deviation, a variance, a range, an interquartile range, moving average value, and an average absolute deviation. Upon calculation of these various values as related to the monitored physiological data, a comparison 38 is made between the monitored physiological data and the reference baseline and trend shifts in the data are measured to see if they are within an acceptable limit.

Various statistical algorithms can be performed by the computer 14 in analyzing and determining these trend shifts. In one embodiment, a double-smoothing algorithm is performed to apply a moving average technique to the time series physiological data acquired. The double-smoothing algorithm compares an average value for the initial physiological data (associated with a specified physiological parameter) to an average value for the monitored physiological data (associated with the same specified physiological parameter). More precisely, the average value of the physiological data acquired during a selected long-term window (i.e., selected lock-in period) is compared to the average value of the physiological data acquired during the short-term window (i.e., diagnosis period). The shift between these average values is examined and compared 40 to a “threshold” or alert limit set by the healthcare professional. The threshold or alert limit, as defined herein, is not a fixed value limit, but takes the form, of at least one of: a difference value between the average values, an absolute difference value between the average values, a rate of change between the average values, and a percentage change between the average values.

It is also envisioned that other statistical algorithms can be performed to analyze shift trends and set alert limits. For example, control charting techniques including non-deterministic algorithms and the use of heuristics can be implemented, as well as t-tests. Additionally, momentum and rate of change algorithms, similar to Moving Average Convergence/Divergence (MACD), can also be implemented. The above described algorithms are exemplary and not not meant to limit the statistical algorithms that can be implemented in determining the physiological data trend shifts, as various other algorithms configured to provide statistical comparisons between time series data can also be implemented. Thus, comparisons of various statistical values, such as the mean, the standard deviation, the variance, the range, the interquartile range, and the average absolute deviation, can be made between the monitored physiological data and the reference baseline.

As set forth above, the threshold or alert limit can take the form of a difference value between the average values of the physiological data from the lock-in period and the diagnosis period, an absolute difference value between the average values, or a percentage change between the average values. If the threshold limit is crossed 42, the computer 14 generates an alarm 44, alerting the healthcare provider of such an occurrence. The healthcare provider is thus able to examine the physiological data acquired from the patient to determine if further action is required, such as bringing the patient in to a healthcare facility for observation and testing.

The threshold and alert limit described above can be used as the sole alert threshold in telemonitoring system 10 (shown in FIG. 1), or it can be used in conjunction with a standard fixed value threshold. That is, while a healthcare provider may wish to be made aware when a patient may be trending into an area of potential medical concern (without having actually crossed a pre-defined fixed threshold), it is also envisioned that the healthcare provider may wish to be alerted when a pre-defined fixed threshold is crossed for a specific physiological parameter. By implementing alerts of both types, a healthcare provider is allowed still greater control and freedom in monitoring a patient.

If no alert is triggered 46 by comparison of the physiological data from the lock-in period and the diagnosis period and if the patient is determined not to be trending toward an area of concern, the computer 14 is configured to incorporate the monitored physiological data into the reference baseline. Incorporation of the newly acquired monitored physiological data into the reference baseline allows for an updated reference baseline to be determined 48, thus keeping up to date the current status of a patient as represented by the updated reference baseline.

As set forth above, a reference baseline is established from initial physiological data acquired during a lock-in period and can further be updated by incorporating monitored physiological data. However, it is also envisioned that initial physiological data acquired during a lock-in period can be insufficient for establishing a reference baseline, as can occur when a patient is started on a new course of treatment that would affect certain measured physiological parameters. For example, up-titration of a blood pressure lowering drug that is administered to a patient would decrease the value of blood pressure readings that are taken, thus necessitating an updated reference baseline that will reflect these lowered values. Therefore, computer 14 (shown in FIG. 1) is further programmed to determine 30 if the initial physiological data is sufficient to determine the reference baseline (based on input provided by the healthcare professional), and if it is not sufficient 50, to receive 52 supplemental physiological data from the monitoring device. Computer 14 sets a reminder 54 for healthcare personnel to review the acquired supplemental physiological data at the end of a supplemental time period during which the data is acquired. A healthcare professional can then review the supplemental physiological data to determine whether the data is sufficient to form an acceptable reference baseline. If the supplemental physiological data is determined to be sufficient, at least a part of the supplemental physiological data is then used for determining the reference baseline. More specifically, a lock-in period or long-term window is selected covering at least a portion of the supplemental time period during which the supplemental physiological data was gathered, and physiological data from this lock-in period is used in determining the reference baseline.

It is also envisioned that a healthcare professional, upon administering a different drug or amount of drug to a patient, may desire to compare data on a physiological parameter from a period prior to administering the drug to a period following administering the new drug. That is, initial physiological data acquired during a lock-in period pre-administration of the drug can be compared to additional physiological data acquired post-administration of the drug. As such, computer 14 can be further configured to compare the additional physiological data to the initial physiological data to determine shifts therebetween, thus determining the effectiveness of the drug.

### EXAMPLE 1

Here below, an example is provided that describes the use of telemonitoring system 10 (shown in FIG. 1) in detecting acute decompensation in a heart failure patient by way of weight telemonitoring. Weight changes are often a symptom of acute decompensation in heart failure patients and monitoring of such weight in an efficient manner is highly desirable. As such, monitoring such weight changes via telemonitoring and tracking trend shifts in the weight gain/loss of a patient can lead to early diagnosis of a pending acute decompensation.

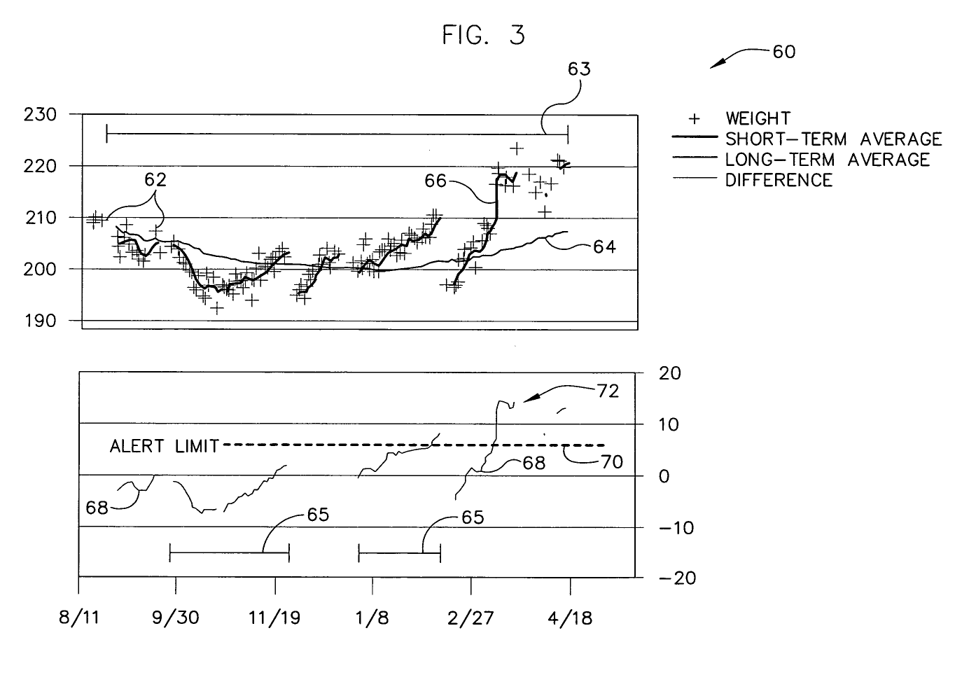

Referring now to FIG. 3, a graph 60 is set forth depicting a weight telemonitoring simulation performed by telemonitoring system 10. Physiological data related to the weight of a patient was acquired via a monitoring device (i.e., a scale), as set forth in detail above. Weight data 62 (i.e., initial physiological data) was acquired in a time series over a long-term window 63. An alerting algorithm was programmed into the computer 14 (shown in FIG. 1) that uses a moving average technique to determine a long-term average value 64 of the weight data obtained in the long-term window 63, the long-term average value 64 of the weight data forming at least part of the reference baseline. Intermittently within the long-term window 63, short-term windows 65 were defined during which weight data 62 (i.e., monitored physiological data) was compared to the long-term average value 64. A separate short-term average value 66 for weight data was determined for each short-term window 65, again by way of the alerting algorithm and a moving average technique performed thereby.

Upon acquisition of both the long-term average value 64 and the short-term average values 66 of the weight data, the alerting algorithm performed a double-smoothing operation comparing the long-term average value of weight and the short-term average values of weight. A difference 68 between the long-term average value 64 and each of the short-term average values 66 was calculated and compared to a designated threshold 70, shown in FIG. 3 as a difference threshold (i.e., alert limit) set at approximately 5 lbs weight gain. In the event of a large difference that exceeded 72 the designated threshold 70, an alert was generated by the telemonitoring system, notifying a healthcare provider of such exceedances.

In performing the alerting algorithm to detect and measure trend shifts and variances of patient weight, numerous criteria can be varied to affect the occurrence of threshold exceedances and both the long-term and short-term average weight values. That is, the length of each of the long-term window and the short-term window impacts the number of threshold exceedances and the long-term and short-term average weight values. As such, the alerting algorithm can be optimized by selecting window lengths that will generate (via threshold exceedances) a greater number of “true” alerts that accurately reflect a pending acute decompensation event, while also lessening the amount of false alerts generated by the telemonitoring system.

A technical contribution for the disclosed method and apparatus is that is provides for a computer implemented process that measures shifts in physiological data by comparing short-term window monitored physiological data to long-term window initial physiological data by way of statistical algorithms and generates an alert for shifts that exceed a pre-determined threshold value.

Therefore, according to one embodiment of the present invention, a telemonitoring alert system for patient care includes a monitoring device configured to acquire physiological data from a patient under observation and a computer in communication with the monitoring device to receive physiological data therefrom. The computer is programmed to receive initial physiological data from the monitoring device during a lock-in period and receive monitored physiological data from the monitoring device during a diagnosis period. The computer is further programmed to measure shifts in the physiological data by comparing the monitored physiological data to the initial physiological data and generate an alert for shifts that exceed at least one of a pre-determined size and rate.

According to another embodiment of the present invention, a method for telemonitoring a patient includes the steps of acquiring reference physiological data from a patient under observation and determining a reference baseline from the reference physiological data. The method also includes the steps of acquiring follow-up physiological data from the patient, comparing the follow-up physiological data to the reference baseline to identify a variation between the follow-up physiological data and the reference baseline, and generating an alert if the variation is outside a pre-determined threshold.

According to yet another embodiment of the present invention, a computer readable storage medium includes a computer program thereon that provides alerts in a patient telemonitoring system. The computer program comprises a set of instructions that, when executed by a computer, causes the computer to receive physiological data on at least one physiological parameter, wherein the physiological data includes a first data set and a second data set. The set of instructions further causes the computer to determine a baseline patient status from at least a portion of the first data set, measure a shift in the second data set from the baseline patient status, and generate an alarm if the shift in the second data set exceeds a pre-determined threshold amount.

The present invention has been described in terms of the preferred embodiment, and it is recognized that equivalents, alternatives, and modifications, aside from those expressly stated, are possible and within the scope of the appending claims.

1. A telemonitoring alert system for patient care comprising:
a monitoring device configured to acquire physiological data from a patient under observation; and
a computer in communication with the monitoring device to receive physiological data therefrom, the computer programmed to:
receive initial physiological data from the monitoring device during a lock- in period;
receive monitored physiological data from the monitoring device during a diagnosis period;
measure shifts in the physiological data by comparing the monitored physiological data to the initial physiological data; and
generate an alert for shifts that exceed at least one of a pre-determined size and rate.

2. The telemonitoring alert system of claim 1 wherein the computer is further programmed to determine a reference baseline from the initial physiological data.

3. The telemonitoring alert system of claim 2 wherein the computer is further programmed to perform an outlier removal to exclude specified initial physiological data in determining the reference baseline.

4. The telemonitoring alert system of claim 2 wherein the computer is further programmed to:
incorporate the monitored physiological data into the reference baseline; and
determine an updated reference baseline from the monitored physiological data and the initial physiological data.

5. The telemonitoring alert system of claim 2 wherein the computer is further programmed to:
determine if the initial physiological data is sufficient to determine the reference baseline;
receive supplemental physiological data from the monitoring device during a supplemental time period if the initial physiological data is not sufficient;
set a reminder for the end of the supplemental time period to determine if the supplemental physiological data is sufficient to determine the reference baseline.

6. The telemonitoring alert system of claim 5 wherein the computer is further programmed to:
compare the supplemental physiological data to the initial physiological data;
determine shifts in the physiological data by comparing the supplemental physiological data to the initial physiological data.

7. The telemonitoring alert system of claim 1 wherein the computer is further programmed to determine at least one of a mean, a standard deviation, a variance, a range, an interquartile range, a moving average value, and an average absolute deviation for each of the initial physiological data and the monitored physiological data.

8. The telemonitoring alert system of claim 7 wherein the computer is further programmed to compare at least one of the mean, standard deviation, the variance, the range, the interquartile range, the moving average value, and the average absolute deviation of the monitored physiological data to at least one of the mean, the standard deviation, the variance, the range, the interquartile range, the moving average value, and the average absolute deviation of the initial physiological data.

9. The telemonitoring alert system of claim 1 wherein the computer is further programmed to select a length of the lock-in period and a length of the diagnosis period.

10. The telemonitoring alert system of claim 1 wherein the physiological data further comprises data related to at least one physiological parameter, wherein the physiological parameters comprises one of blood pressure, weight, pulse, and saturation of peripheral oxygen (Spo2).

11. The telemonitoring alert system of claim 1 wherein the computer is further programmed to define the at least one of a pre-determined size and rate as at least one of a percentage change, a rate of change, and a difference value.

12.-22. (canceled)

23. A monitoring and determining system comprising:
a monitoring device configured to measure at least one physiological parameter from a patient;
a computer in communication with the monitoring device to receive one of the at least one physiological parameter and physiological data therefrom, the computer programmed to:
receive a lock-in period selection;
acquire physiological data from the monitoring device for the selected lock-in period;
establish a reference baseline for the patient based on the acquired physiological data;
receive a diagnosis period selection;
acquire physiological data from the monitoring device for the selected diagnosis period;
perform at least one statistical algorithm on the acquired physiological data for the selected diagnosis period, thereby to provide statistical analysis of the acquired physiological data, wherein the at least one statistical algorithm is one of mean, standard deviation, variance, range, interquartile range, moving average value, and average absolute deviation;
compare the physiological data for the lock-in period to the statistical analysis of the physiological data for the diagnosis period;
generate an alarm when a threshold limit is crossed, based on the comparison.

24. The system of claim 23, wherein the diagnosis period is shorter than the lock-in period.

25. The system of claim 23, wherein the threshold limit is at least one of: a difference value between average values, an absolute difference value between average values, a rate of change between average values, and a percentage change between average values.

26. A system for patient care comprising:
a computer configured to receive at least one physiological parameter from one of a patient and a monitoring device, the computer programmed to:
receive a lock-in period selection;
acquire physiological data for the selected lock-in period;
establish a reference baseline for the patient based on the acquired physiological data;
receive a diagnosis period selection;
acquire physiological data for the selected diagnosis period;
perform at least one statistical algorithm on the acquired physiorogical data for the selected diagnosis period, thereby to provide statistical analysis of the acquired physiological data, wherein the at least one statistical algorithm is one of mean, standard deviation, variance, range, interquartile range, moving average value, average absolute deviation, moving average convergence/divergence, and non-deterministic algorithms;
compare the physiological data for the lock-in period to the statistical analysis of the physiological data for the diagnosis period;
generate an alarm when a threshold limit is crossed, based on the comparison, wherein the threshold limit is at least one of: a difference value between average values, an absolute difference value between average values, a rate of change between average values, and a percentage change between average values.
